(19) United States
(12) Patent Application Publication
(10) Pub. No.: US 2017/0188604 A1
(43) Pub. Date: Jul. 6, 2017

(54) ENZYME FORTIFIED FOODS; ADD-ONS, SUSPENDED AND COATED PROCESSES

(71) Applicants: Xiaohong Zhang, Clayton, MO (US); Michael M. Saigh, Clayton, MO (US)

(72) Inventors: Xiaohong Zhang, Clayton, MO (US); Michael M. Saigh, Clayton, MO (US)

(21) Appl. No.: 14/989,238

(22) Filed: Jan. 6, 2016

(51) Int. Cl. A23K 20/189; A61K 38/43

(52) U.S. Cl. CPC A23L 1/305; A23K 20/189; A61K 38/43; A23V 2002/00

(57) ABSTRACT

Typical digestive enzymes for humans not produced by the body or naturally occurring in foods are provided by SEP, supplements. In many cases the body does not have the capacity of producing the quantity of both digestive and metabolic enzymes as they are needed as described in the above background. People when eating out or traveling forget to pack the enzymes when eating at a restaurant. SEP though available to consumers as orally taken supplements before and after a meal; are often are not taken in a timely manner due to the forgetfulness, lack of supply and or convenience. The current invention could be defined by one embodiment, as the ability to help humans, pets and animals to ingest one or more enzyme source fundamentally embedded in the foods they actually eat and consume. In other words, Food from all food groups as shown in FIG. 3 (Food Pyramid) broken down into its nutrient pieces: amino acids (from proteins), fatty acids and cholesterol (from fats), and simple sugars (from carbohydrates), as well as vitamins, minerals, and a variety of other plant and animal compounds.

A major embodiment of the present invention is based on the concept of Enzyme Fortified Foods (EFF). EFF is defined as one or more enzymes are fundamentally rooted and or systemically embedded to one or more food categories, and/or food classification(s) and/or good groups and/or food recipes and/or food sources and/or food uses etc. EFF is not administered by the same means as SEP dosages. SEP dosages are administered in the form of pills, capsules and or chewables. EFF's are administered by adding or mixing one or more enzymes directly to the digested food source.

In addition to digestive enzymes, the present invention includes but is not limited to all types and categories of enzymes for all metabolic functions and benefits. For specifically, the new system could include various methods enhancing enzyme, one scenario could include for example, the following: Digestive enzymes, primarily produced in the pancreas and small intestine, break down our food into nutrients so that our bodies can absorb them.

The present invention builds a new brand category (Enzyme Fortified Foods—“EFF”) for all food manufactures and all food processors, restaurants, and food handlers (commercial and home) which could add one or more enzyme which optimizes digestion regarding a specific product and or product line.

The invention includes a system of measurements based on the type of enzyme as listed in Enzyme Catalog A. It is understood by anyone familiar with the art that one or more classification of enzymes could be combined as listed in Enzyme Catalog A. One or more Enzymes could be added to any and all NAICS food groups (The North American Industry Classification System (NAICS) is the standard used by Federal statistical agencies in classifying business establishments for the purpose of collecting, analyzing, and publishing statistical data related to the U.S. business economy). It is understood by anyone familiar with the art that all food groups in all countries around the world could be categorized with a similar system.

### CROSS-REFERENCE TO RELATED APPLICATIONS

This application is a provisional application, and claims the benefit of the filing date, of Jan. 6, 2015, the teachings of which are incorporated herein in their entireties by reference.

### BACKGROUND OF THE INVENTION

Field of the Invention

The present invention relates to calculable enzyme addition in order to enhance digestion for both animals and humans.

Description of the Related Art

Enzymes are biological catalysts or assistants. Enzymes consist of various types of proteins that work to drive the chemical reaction required for a specific action or nutrient. In biology, one of the factors that define a living thing from an inanimate object is the organism's ability to carry out chemical reactions that are crucial for its survival. Enzymes are defined as any of various proteins, as pepsin, originating from living cells and capable of producing certain chemical changes in organic substances by catalytic action, as in digestion. If our digestive system doesn't have enough digestive enzymes, or bodies can't break down our food and as result of digestive system can't absorb the needed good nutrition required for health and longevity. When digestion does not occur the way it should, symptoms such as gas, bloating, and pain may result.

Both humans and animals are capable of thousands of chemical reactions within their bodies. Enzymes are specialized proteins built in the body to accomplish a variety of tasks. In the metabolism, enzymes help the body break down, transport, and transform food products people ingest. They are found in the digestive tract, liver, kidneys, and other structures of the body and are involved at every level, from cellular metabolism to the gross breakdown of chemical compounds processed in the liver.

None of these reactions are possible without enzymes. Enzymes work on substrates (a substance in which the enzyme acts) in one of three ways; physical stress (PS), substrate orientation (SO), and changes in substrate reactivity (SR). SO occurs when an enzyme causes substrate molecules to align with each other and form a bond. When an enzyme uses (PS) on a substrate, it in effect grips the substrate and forces the molecule to break apart. An enzyme that causes changes in substrate reactivity (SR) alters the placement of the molecule's electrons, which influences the molecule's ability to bond with other molecules.

The catalytic properties of enzymes are acyclic process. Enzymes have active sites where they come into contact with particular substrates. When the substrate has come into contact with the active enzyme, it is modified by the enzyme to form the end product. The process is complete; the enzyme releases the product and is ready to begin the process with new substrates.

Enzymes can speed it up. The chemicals that are transformed with the help of enzymes are called substrates. In the absence of enzymes, these chemicals are called reactants.

Enzymes can speed up and or launch a reaction and efficiencies. There are an estimated 75,000 different enzymes in the human body and in most advanced animal bodies.

However, enzyme deficiency is genetic condition characterized by inability to produce an enzyme or underproduction of that enzyme, leading to health problems caused by problems with the metabolism. Some examples of enzyme deficiencies include phenylketonuria, acute intermittent porphyria, alkaptonuria, and Lesch-Nyhan syndrome, etc.

When there is an enzyme deficiency, not enough of an enzyme is produced by the body. This could be the result of a genetic error where the code for producing the enzyme is not present or is distorted. Enzyme deficiency may lead to a variety of health problems. Patients may experience buildups of toxins in the body, declines in organ function, and other issues.

Digestion

Digestive enzymes are enzymes that break down food into usable material. The major different types of digestive enzymes are which include, but limited to any one of a combination of more than one from the following as listed in Enzyme Catalog A.

### Enzyme Catalog A

amylase—breaks down carbohydrates, starches, and sugars which are prevalent in potatoes, fruits, vegetables, and many snack foods
lactase—breaks down lactose (milk sugars)
diastase—digests vegetable starch
sucrase—digests complex sugars and starches
maltase—digests disaccharides to monosaccharides (malt sugars)
invertase—breaks down sucrose (table sugar)
glucoamylase—breaks down starch to glucose
alpha-glactosidase—facilitates digestion of beans, legumes, seeds, roots, soy products, and underground stems
protease—breaks down proteins found in meats, nuts, eggs, and cheese
pepsin—breaks down proteins into peptides
peptidase—breaks down small peptide proteins to amino acids
trypsin—derived from animal pancreas, breaks down proteins
alpha—chymotrypsin, an animal-derived enzyme, breaks down proteins
bromelain—derived from pineapple, breaks down a broad spectrum of proteins, has anti-inflammatory properties, effective over very wide pH range
papain—derived from raw papaya, broad range of substrates and pH, works well breaking down small and large proteins
lipase—breaks down fats found in most dairy products, nuts, oils, and meat
cellulase—breaks down cellulose, plant fiber; not found in humans
betaine HCL—increases the hydrochloric acid content of the upper digestive system; activates the protein digesting enzyme pepsin in the stomach (does not influence plant- or fungal-derived enzymes)
endoprotease—cleaves peptide bonds from the interior of peptide chains
exoprotease—cleaves off amino acids from the ends of peptide chains
extract of ox bile—an animal-derived enzyme, stimulates the intestine to move
fructooligosaccharides (FOS)—helps support the growth of friendly intestinal microbes, also inhibits the growth of harmful species
L-glutamic acid—activates the protein digesting enzyme pepsin in the stomach
lysozyme—an animal-derived enzyme, and a component of every lung cell; lysozyme is very important in the control of infections, attacks invading bacterial and viruses
papayotin—from papaya
pancreatin—an animal-derived enzyme, breaks down protein and fats
pancrelipase—an animal-derived enzyme, breaks down protein, fats, and carbohydrates
pectinase—breaks down the pectin in fruit
phytase—digests phytic acid, allows minerals such as calcium, zinc, copper, manganese, etc. to be more available by the body, but does not break down any food proteins
xylanase—breaks down xylan sugars, works well with grains such as corn and other grains, seeds and nuts.

It is understood that anyone familiar with the art that one enzyme or mix of one or more enzymes could be combined from Enzyme Catalog A. It is also understood that other enzymes could be listed beyond the above and/or a combination or new enzymes discovered, categorized and named.

### Enzyme Temperature Variance

Enzymes have a certain shelf life when used to separate out and mix when needed.

Actually, there are enzymes that naturally work at very low temperature even in ice cream. However, these are specific enzymes and not the ones we normally think of for feed or food. In general, however, enzymes work optimally at warm temperatures around 37 C.° or so (between 25-40 C.°). At lower temperatures, they have lower activity and at higher temperature they also have lower activity. Once the temperature gets too high, it denatures their structure (they are made of protein) and therefore renders them inactive permanently. However, in lower temperatures, they are just inactive, unless the temperature goes down too low (Below 0° F.) where the protein structure once again gets destroyed and the enzymes are rendered inactive.

Enzymes typically are heat sensitive, unless they are genetically engineered to withstand high temperature such as Phytaverse (Melting temp is 91.4 C). There are different ways to stabilize the enzymes and one of these are glycosylation using glucose based material, which will bind to the enzyme and give it higher heat stability, another is fat coating, protecting the enzyme from heat as well. All of which are referred to in general as coating, or encapsulation. There are few technologies to do so, but in general, such enzymes are still heat tolerant above 90-95 C and cannot stand extrusion temperatures. However, temperature resistant hybrid enzymes are now being tested to withstand hot temperatures (over boiling) and extreme cold temperatures. In addition, PH is important to the digestion as described by the following”

### Enzyme PH Variance

Hydrochloric acid cannot be adequately produced, as may be the case with older adults, enzymes do not activate. Digestion, or hydrolysis, is the process of breaking up food “into simpler chemical compounds by means of hydrolyzing enzymes or chemical action. In other words, digestion breaks up food into small units so the body can use the nutrients for energy or growth and repair. Hydrochloric acid does not digest food, enzymes digest food. The normal digestive process begins in the mouth. While you chew food, your salivary glands secrete digestive enzymes that begin to work immediately. Enzymes start with for example, when saliva liberates the sugar in the bread or other substances when in the mouth and throat. Raw food is eaten; the naturally occurring enzymes in that food will also participate. If the food enzymes have been denatured or destroyed through a food processing method, only the enzymes in the saliva are available to begin digestion. Even after being chewed, some food pieces are still too large to pass through the wall of the stomach and intestines. They must be broken down into much smaller pieces.

Other symptoms that suggest you might have problems with digestive enzymes are:
Gas and bloating after meals
Indigestion
Feeling full after eating food
Undigested food in the stool
Floating stools
Undigested fat(s)

Enzymes are required to digest all foods; however, as we age the effectiveness of enzymes decrease. In addition, process foods further stress the digestive system and inhibits the natural body's ability to produce and or utilize enzymes for the digestive process.

When you swallow food, the stomach begins to produce the acid and enzymes needed for digestion. This window of time, while the body gathers hydrochloric acid in the stomach, can last for up to one hour. In the meantime, the salivary enzymes and food enzymes are continuing to work at breaking down the ingested food.

As hydrochloric acid gathers in the stomach, it becomes an acidic environment. This acidity is ideal for the activation of the protein-digesting enzyme, pepsinogen, which upon activation becomes pepsin. These protein-digesting enzymes begin working while the food and salivary enzymes are deactivated, since this environment is not optimal for certain enzymes to work. Moreover, these enzymes supplemental and naturally occurring are not destroyed. Based on the amount and type of food you have eaten, the body produces the exact amount of enzymes needed to continue digesting the food.

After the food leaves the stomach, it enters the upper part of the small intestine, called the duodenum. It is here that the liver and gallbladder adds bile to the mix, making the intestinal environment alkaline. Bile does not contain enzymes and does no digestive work. However, it is responsible for degreasing food. In other words, bile acts as a detergent and breaks down any fats or oils that are coating the food. This step is necessary so that the enzymes from the pancreas can reach the food and finish digestion.

The enzymes that work in an alkaline environment are activated, such as supplemental pancreatic enzymes (derived from animals). The body has done most of the work by this time; therefore, supplemental pancreatic enzymes do not necessarily help with digestion before this point. The digested food particles now pass through the intestinal wall to be assimilated by the body. The indigestible portions of the food, such as insoluble fiber, are pushed into the colon for elimination. When one step of the process does not happen, the subsequent digestive actions are affected. For instance, if you take antacids, you are reducing your body's ability to produce hydrochloric acid and, therefore, your ability to digest protein, which now has no way of being thoroughly broken down. Protein digestion must begin with the acidify the stomach, large intestine and small intestine.

However, enzyme adsorption and/or absorption might in the absence of any obvious disease, things still may not be working properly; for some of the following reasons:
Low-grade inflammation, in the digestive tract (such as that caused by “food allergies,” intestinal permeability, dysbiosis, parasitic infection, etc.) can lead to deficiencies in digestive enzymes.
Aging, has been associated with decreased digestive function.
Low stomach acid, increases the likelihood that you won't have adequate digestive enzymes either.
Stress, is the most common reason for digestive enzyme problems. Our body has two modes: sympathetic and parasympathetic. When in the sympathetic mode, digestive is given a very low priority, which means digestive function (including digestive enzyme output) is lowered.

People and animals eat food, but our digestive system doesn't absorb food, it absorbs nutrients and enzymes are necessary for the nutritional process.

Furthermore, most cooked and processed foods lack enzymes. The body produces digestive and metabolic enzyme when needed. Raw foods contain natural enzymes which serve to help digest and breakdown its own matter when consumed. Cooked foods (approximately, over 91° C.) destroys all enzymes. Most of the foods we eat are processed and cooked resulting in an overload for the body to digest the foods unless supplemental enzymes in the form of Supplemental Enzyme Pills (SEP) which could be in the form of not only pills but capsules, chewable tablets and dry mixes). SEP products are currently ready available throughout the world.

Currently, approximately 80% of SEP are plant-based such as (papain, bromelain etc.) and the other 20% are animal-based such as (trypsin, pancreatin and chymotrypsin, etc.). Today's highly processed food culture and food suppliers, maximizing nutrient adsorption and/or absorption takes a back seat to fast convenient. Sympathetic mode of digestion includes a lifestyle of people which aren't giving a high priority to properly digesting our food.

### SUMMARY OF INVENTION

The following summary of the invention is provided and intended to facilitate an understanding of some of the innovative features unique to the invention, and is not intended to be a full description of variations that may be apparent to those of skill in the art. A full appreciation of the various aspects of the invention can be gained from the entire specification, claims, and drawings, abstract and claims taken from the whole.

In summary, the problems with SEP are as follows;
A. Typical digestive enzymes for humans not produced by the body or naturally occurring in foods are provided by SEP, supplements.
B. In many cases the body does not have the capacity of producing the quantity of both digestive and metabolic enzymes as they are needed as described in the above background.
C. In many cases people forget taking enzyme capsules or tablets prior to eating a meal or after eating a meal or snacks. People when eating out or traveling forget to pack the enzymes when eating at a restaurant. SEP though available to consumers as orally taken supplements before and after a meal; are often are not taken in a timely manner due to the forgetfulness, lack of supply and or convenience. The current invention could be defined by one embodiment, as the ability to help humans, pets and animals to ingest one or more enzyme source fundamentally embedded in the foods they actually eat and consume. In other words, Food from all food groups as shown in FIG. 3 (Food Pyramid) broken down into its nutrient pieces: amino acids (from proteins), fatty acids and cholesterol (from fats), and simple sugars (from carbohydrates), as well as vitamins, minerals, and a variety of other plant and animal compounds.

### Enzyme Fortified Foods (EFF)

A major embodiment of the present invention is based on the concept of Enzyme Fortified Foods (EFF). EFF is defined as one or more enzymes are fundamentally rooted and or systemically embedded to one or more food categories, and/or food classification(s) and/or good groups and/or food recipes and/or food sources and/or food uses etc. EFF is not administered by the same means as SEP dosages. SEP dosages are administered in the form of pills, capsules and or chewables. EFF's are administered by adding or mixing one or more enzymes directly to the digested food source. There are Three Major EFF Categories:
1. ADD-ON; EFFs example could be in the spices and herbs could be mixed with various EFFs when added to meat after the cooking process; other spices and herbs could be mixed with other EFFs when added to vegetables, snacks, pizza, cooked meats, fish, poultry, fruits, etc.
2. SUSPENDED; EFF examples such as frozen goods, beverages, dairy, dehydrated foods, condiments and sauces, (oil, fat, salt, sugar), herbs, spices in a suspended substance, etc.
3. COATED, EFF formulated to coat various enzymes through the food manufacturing process without destroying the enzymes. Some examples could be cereals, snacks, flavorings and animal foods, etc.

Enzymes are considered a food in itself and derived mostly from plants. After years of research there has not been one report or side effects from over dosing from enzyme consumption. A major distinguishing difference between SEPs and EFFs is the method of administering enzymes. SEPs are based on orally taking pills, capsules and chewables, EFFs are systemic food based additive. EFF intake is based on holistic food consumption. EFF dosage administration are linked to various foods, food groups and classifications. If EFFs are not linked to the food item, the person or animal will not intake enzymes when consuming the food or meal. In addition, a meal could consist of many food groups, when consumed if EFF fortified enzyme intake is an incorporation of all.

In addition to digestive enzymes, the present invention includes but is not limited to all types and categories of enzymes for all metabolic functions and benefits.

Percent Daily Value (DV) in the amount of servings (milligrams, grams) pills, capsules or chewables. EFFs could be calibrated to a person's (animals) diet, food intake, caloric consumption, medical and/or metabolic requirements. EFFs as described in the present invention are linked to a person's food intake as a whole and union of various enzymes based on the food itself. It is understood by anyone familiar with the art that that EFFs could be customized to any diet, customized to any food classification, cuisine, combination of intake, or method of fighting any digestive or other disease.

For specifically, the new system could include various methods enhancing enzyme, one scenario could include for example, the following:

Digestive enzymes, primarily produced in the pancreas and small intestine, break down our food into nutrients so that our bodies can absorb them.
Adsorption is the adhesion of atoms, ions, or molecules from a gas, liquid, or dissolved solid to a surface. This process creates a film of the adsorbate on the surface of the adsorption.
Absorption (chemistry), absorption of particles of gas or liquid in liquid or solid material
Absorption (skin), a route by which substances can enter the body through the skin
Absorption (pharmacokinetics), absorption of drugs in body

However, the present invention helps correct these necessary dietary constraints in that the enzymes are fortified as an add-on to all process foods. These foods could include any and all food groups in including but limited to the following list described in the Grocery and Animal Product Classification List B and as it relates to one or more enzymes as listed in Enzyme Catalog A. In addition

### Grocery and Animal Product Classification List B.

Meat, Fish, Poultry
Dairy
Dehydrated
Bakery
Canned Goods
Bottled Goods
Plastic Container Goods
Boxed Goods
Spices and Herbs
Frozen Foods
Drinks and Beverages
Fruits & Vegetables
Animal Feed
Livestock Feed

In addition, other food categories include but not limited to: all packaged goods and foods, deli and restaurant items, wholesale, commercial and manufactured foods, homemade meals, institutional meals, confectioneries, concessions, toppings and condiments, all other foods, feeds and product categories listed in Product Classification List B as it relates to all enzyme classifications.

The present invention builds a new brand category (Enzyme Fortified Foods—“EFF”) for all food manufactures and all food processors, restaurants, and food handlers (commercial and home) which could add one or more enzyme which optimizes digestion regarding a specific product and or product line.

In addition, in another embodiment, various foods as exemplified by the following;

### Possible Items for Enzyme Enhanced Products:

Cocoa, Sugar (cane, brown, powdered) flour, Spices, 100's of possibilities (Including for meats and or veggie mixes), cereals, snacks, nuts, popcorn, candy; chocolate, bars, sweets, etc.), gum, baby foods, baby formula, whole or grated, cheeses, salad dressing and seasoning mix packets etc.

All prepared dry or seasoning mixes, without heating required or for example exceeded the temperature capable of killing the enzyme or mix of food products and enzymes. The addition of enzyme in the manufacturing or assembly process could be timed when adding enzymes. For example, after the mix is cooled below the temperature of less than 140° F. or added prior to packaging the item or serving the item.

An example of adding a “EnZymed Fortified Foods” (EFF) enhancement to a frozen pizza could take place prior to serving the pizza. A container of parmesan cheese with a measured amount mixed enzyme or a container of spice with a measured amount of mixed enzyme could be sprinkled on top of the pizza prior to serving.

It is understood by anyone familiar with the art that EnZymed enhancement and or added enzyme fortification could be added to any grocery and food category during the time its processes and or after the time the item is processed.

“Enzyme Fortified” “Enzyme Enhanced” Statement/Claim on Product Enzyme Introduction Process and implementation can be of any type of: Coating, Mixture, Additive, Embedded, Enhancement, Seasoning, Sprinkling, Flavoring, Other.

The invention includes a system of measurements based on the type of enzyme as listed in Enzyme Catalog A. It is understood by anyone familiar with the art that one or more classification of enzymes could be combined as listed in Enzyme Catalog A. One or more Enzymes could be added to any and all NAICS food groups (The North American Industry Classification System (NAICS) is the standard used by Federal statistical agencies in classifying business establishments for the purpose of collecting, analyzing, and publishing statistical data related to the U.S. business economy). It is understood by anyone familiar with the art that all food groups in all countries around the world could be categorized with a similar system.

It's also understood; the food groups could be combination of food elements or combined food elements in any formula or recipes to make one or more final food products.

In addition, any combination of the Enzyme Catalog A could be combined to better match the type of food ingested by either human or animal in order to help digestion. For example: protease, pepsin, peptidase could be added to meat as spices after cooking process, to assist in the digestion of meat (lamb, beef, poultry, bison) and other protein based food. Another example could be addition of specific Enzyme, such as diastase, surcease and glucoamylase, to help breakdown starch and carbohydrate, such as bread. EFF could be specifically formulated to not only breakdown carbohydrate and starches as exemplified above but also could be combined with Enzymes to digest meat.

For example, if person consumes a hamburger, the hamburger consists both starches and carbohydrate from bread and ground beef. Thus protease, pepsin, peptidase, diastase, surcease and glucoamylase can work together to help digest system as a whole. There is a multitude of thousands combination can be presented in the present invention, depending on what food or combination of foods are digested, what quantities are digested.

It's also understood for anyone who familiar with the art, EFF could improve other functions besides digestion for both humans and animals. Some of which are, but not limit to, eye sight, various internal organs, and muscularity, etc.

In addition as stated above, the present invention includes a new brand category, we refer to as EFF. The addition EFF (Enzyme Fortified Foods) provide immediately intake of a small amount of specific enzyme digested simultaneously with the consumed products. In another words, the food products will include one or more enzymes when absorbed starting with the chewing process.

It's understood for anyone who familiar with the art, the type of one or more enzymes could be used for any product and/or sku as classified shown in the GROCERY AND ANIMAL PRODUCT CLASSIFICATION LIST B.

Determining the type of enzyme or enzymes could be formulated to the type of product. For example; a beef meat product verses a poultry product. The enzyme mix could be specifically formulated to maximize the digestion of the beef dish when another type of enzyme could be formulated for poultry.

In another example, a fresh deli product could most effectively use certain enzymes which hold up better in subzero or minus Celsius temperatures.

The invention in yet another embodiment could analytically compare the product's ingredients, including but not limited to types and/or amounts of proteins carbohydrates, fats (total and trans), salts, oils, preservatives, and vitamins, etc. The enzyme additive mix could be adjusted to optimize a person or animal's digestive ability based on or more food sources consumed for meals and snacks.

### System of Measurements

Many food brand labels contains the number of serving size: for example; ¼ tsp. (0.07 g) as a percent of Daily Values (DV) based on a certain number of consumed calories. Serving units, for example, about 273 for a seasoning blend of a total NET WT of 6.75 OZ (191 g) could be one calibration of measurement used when adding enzymes to a plurality of different food products, food groups and classifications, food combinations. It is understood by anyone familiar with the art that food containers, bottles, cartons, bags and any container could be used to add enzymes to any food or combination of foods.

EFF added to various food groups act in similar fashion to capsules, chewables, supplements;

For example, digestive enzymes work well for some people, but tend to be the weakest digestive enzyme supplement, and aren't sufficient for people who need more support. Animal sourced (typically listed as pancreatin) are not for vegetarians or vegans, and can have issues with stability. “Plant” sourced (from fungus) are the most stable of all the enzymes, survive digestion well, and have a broad spectrum of action.

Dosage and measurement could also vary based on service units based on the number of serving's consumed. It's necessarily the intent to replace with the natural enzyme produced by the body. It's also understood by the present invention that each individual body and metabolism is unique and could require different combination of EFF, strengths and amounts. An individual's physician should ultimately advise his/her patient with the consumption of various food groups, dietary plan, as well as the amount and type of Enzymes required for optimal health. Furthermore, it's understood that age, current health condition, personal activities level, food habits, environment and other criteria could affect the result of EFF intake, as suggested by National Digestive Enzyme Council.

The addition of EFF:
1. Enriched with enzymes can help to restore normal digestive function, including digestive enzymes.
2. Reduce inflammation in the body and the digestive tract, improving nutrient deficiencies, removing enzyme inhibitors. Improper digestion is another issue that diet alone may not solve.
3. Increase enzyme supplementation necessary to help your body properly break down your food.

Timing of taking Enzyme is critical, EFF solves the timing issue by attached food source with Enzyme in a form of coating, suspension and or add-on. A major embodiment of the present invention of the creation of enzyme enhanced (enriched foods) Enzyme enhanced foods through, coatings, flavorings, suspension enzyme additives, mixtures etc. are generally digested when needed during the eating process.

### BRIEF DESCRIPTION OF THE DRAWINGS

Other aspects, features, and advantages of the present invention will become more fully apparent from the following detailed description, the appended claims, and the accompanying drawings in which like reference numerals identify similar or identical elements.

FIG. 1 shows a diagram of Enzyme activities in digestive system, in accordance with embodiments of the present invention;

FIG. 2 shows a logical diagram of major digestive enzyme produced by the body, in accordance with embodiments of the present invention;

FIG. 3 shows a block diagram of food pyramid and daily serving of each food group, in accordance with embodiments of the present invention;

FIG. 4 an exemplary of suspended enzyme food classification, in accordance with embodiments of the present invention;

FIG. 5 shows an exemplary of Spices, Herbs, Seasoning and Flavoring Application, either in separated and or combined dispenser, in accordance with embodiments of the present invention;

FIG. 6 shows a design of enzyme been added into the food manufacturing process, in accordance with embodiments of the present invention;

FIG. 7 shows an exemplary of processed or manufactured food with enzyme coating, in accordance with embodiments of the present invention;

FIG. 8 shows a table of Toppings, Condiments, Sauces & Spices, etc. in accordance with embodiments of the present invention;

FIG. 9 shows an exemplary of Enzyme enhanced product categories, in accordance with embodiments of the present invention;

FIG. 10 shows an exemplary of Enzyme enhanced food product label, in accordance with embodiments of the present invention;

### DETAILED DESCRIPTION

Table 1 defines a list of acronyms employed throughout this specification as an aid to understanding the described embodiments of the present invention:

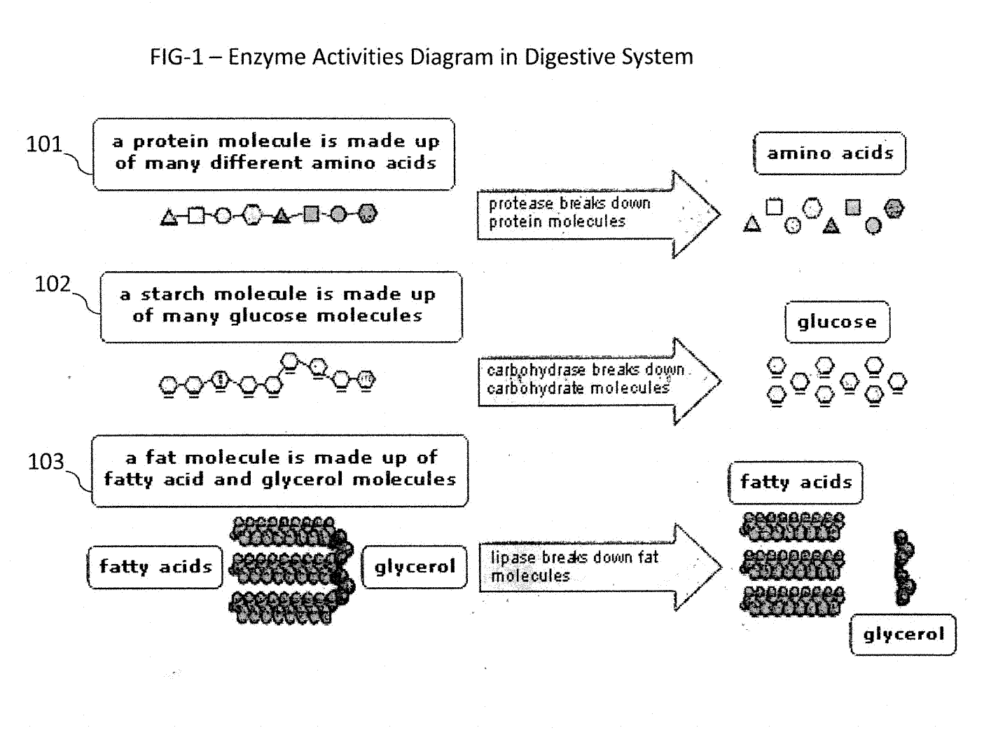

FIG. 1 exemplifies a diagram of various Enzyme activities in digestive system, such as 101—Protease can breakdown protein molecules into amino acid, 102—carbohydrase breaks down carbohydrate molecules into glucose, 103—lipase breaks down fat molecules into fatty acid and glycerol.

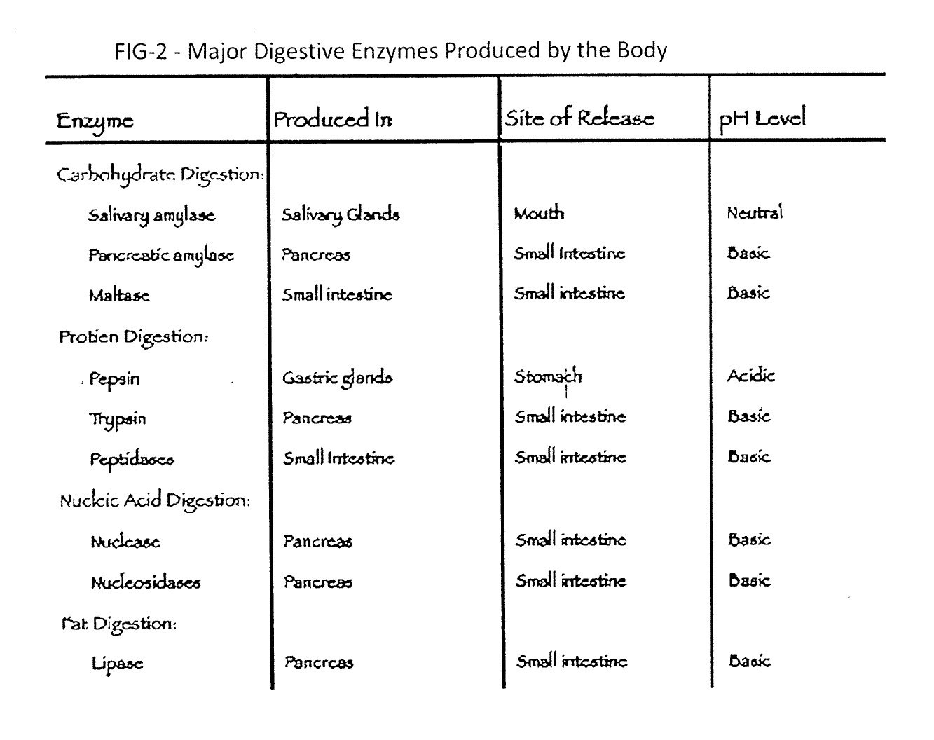

FIG. 2 exemplifies major digestive enzymes produced by the body with various PH level. For example, carbohydrase enzyme (salivary emylase) produced in human mouth with neural PH. Due to health condition and human food intake, the body might not produce enough of enzyme to support daily activities. Thus regular enzyme intake will help to improve enzyme in the body.

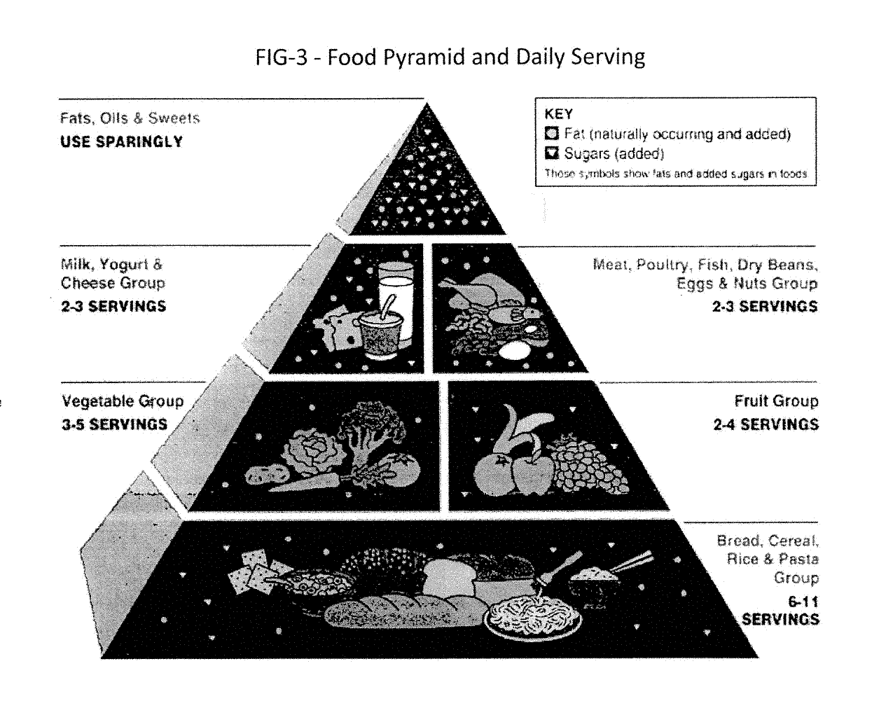

FIG. 3 exemplifies a diagram of food pyramid and daily serving of each food group, in accordance with embodiments of the present invention. In addition, intake of certain mineral could enhance enzyme activities, such as magnesium is crucial to more than 300 enzyme-driven biochemical reactions occurring in the body on a near constant basis.
All nutrients used by the human body function as either:
Sources of energy
Building blocks for body structures
Elements needed to regulate and control the body's many functions

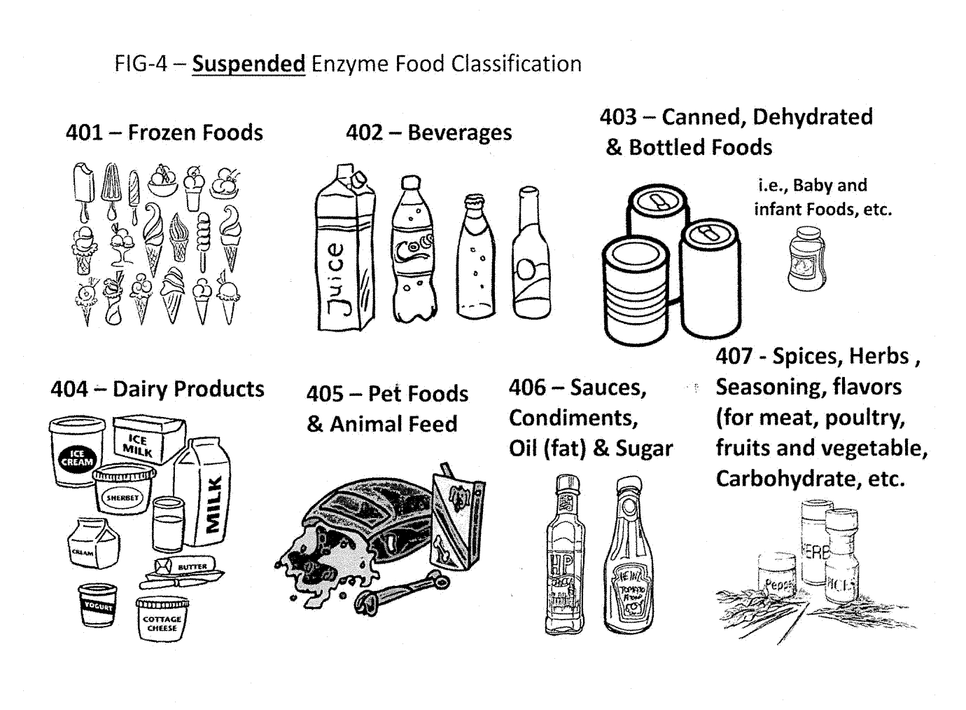

FIG. 4 an exemplary of suspended enzyme food classification, in accordance with embodiments of the present invention; such as 401—Frozen food group, 402—Beverage, 403—Canned, dehydrated and bottled food group include baby and infant products, 404—Dairy product group, 405—Pet food and animal feed, 406—Sauce, condiment, oil (fat) and sugar, 407—Spices, Herbs, Seasoning, flavors for meat, poultry, fruits and vegetable, Carbohydrate, etc.

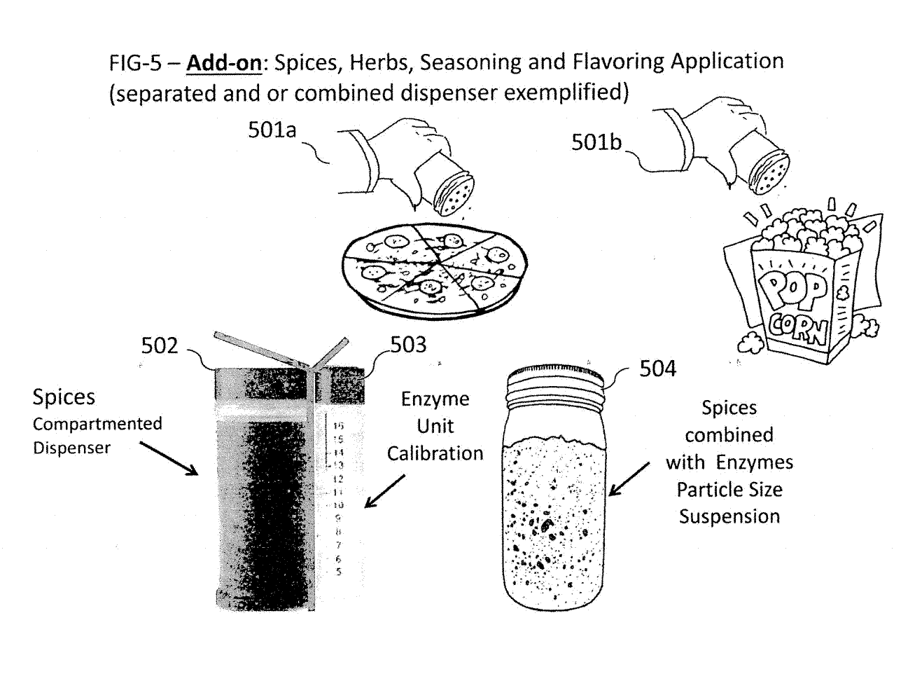

FIG. 5 exemplifies an exemplary of Spices, Herbs, Seasoning and Flavoring Application, either in separated and or combined dispenser, in accordance with embodiments of the present invention; such as, 501—shows spices adding to the food after finished cooking. 502 & 503—shows separate compartmental dispenser with season (502) and enzyme (503), which will allow adding enzyme with preferred amount. 504—shows spices combined with enzyme, and user will not have to worry about serving or adding enzyme separately.

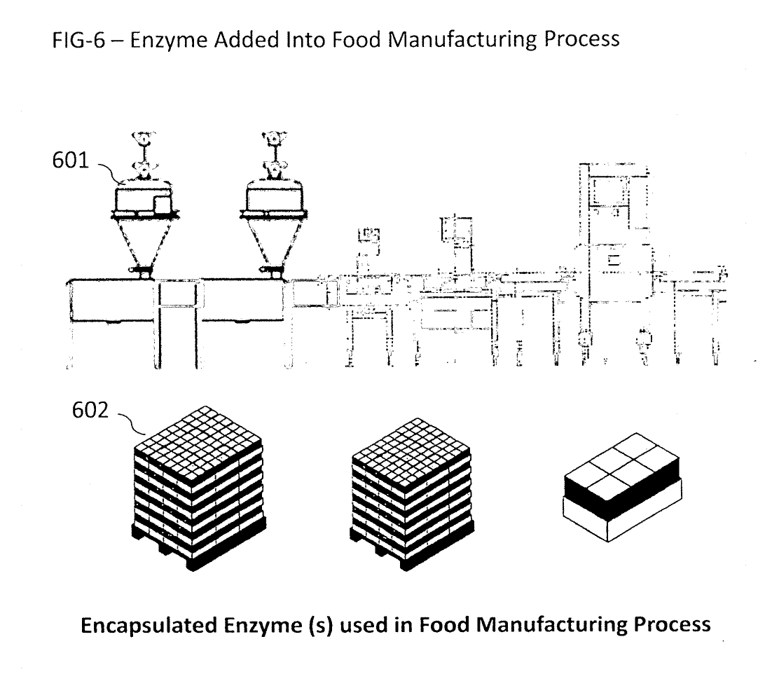

FIG. 6 exemplifies a design of enzyme been added into the food manufacturing process, including but not limit to encapsulated enzyme for the heating process which include all and any processed food for commercial, industrial and retail. 601—shows Enzyme been added at the end of finishing process, include but not limit to blending, mixing, coating, etc. 602—represents the finished EFF products for all food production.

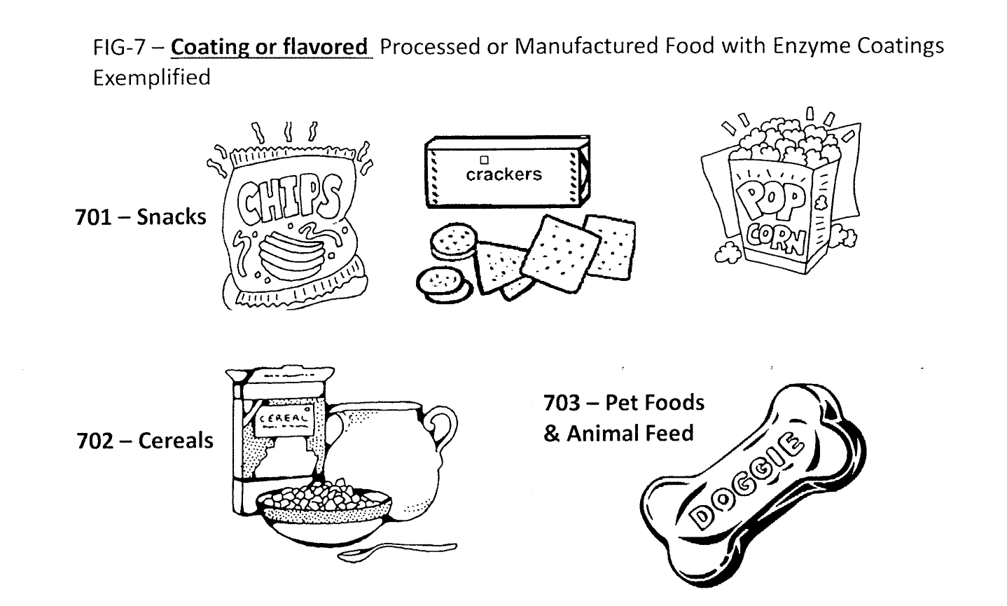

FIG. 7 shows an exemplary of processed or manufactured food with enzyme coated or blended, such as 701—snacks food: chips, crackers, popcorns, etc. 703—Enzyme coating or blends added to cereals. 703—Enzyme through coating and or various blends added to pet or animal food.

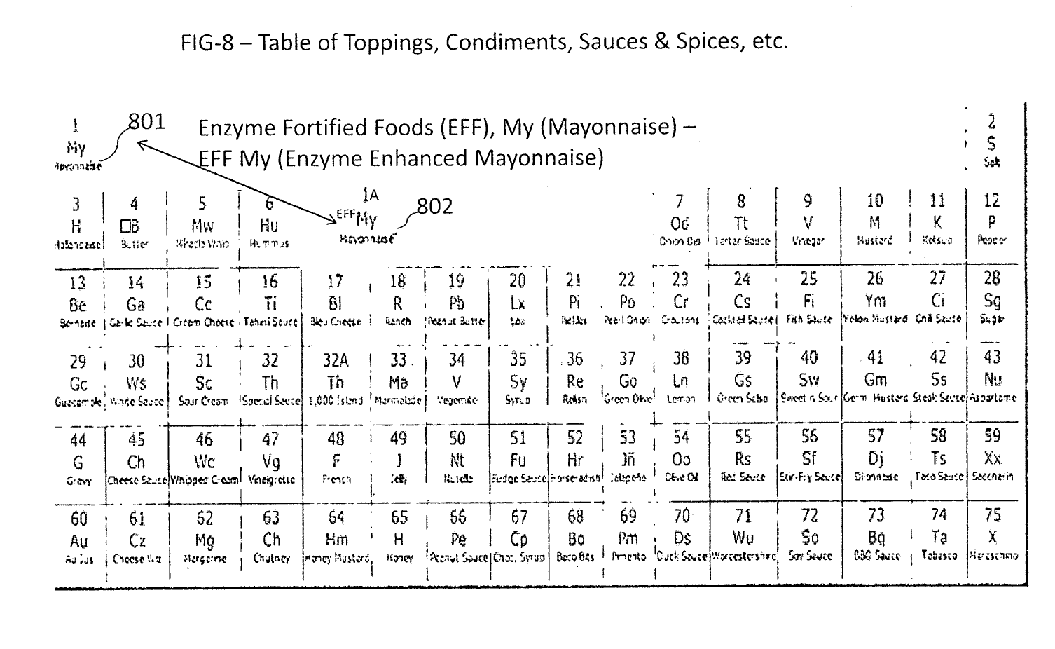

FIG. 8 shows a table of Toppings, Condiments, Sauces & Spices, etc. in accordance with embodiments of the present invention; The Toppings, Condiments, Sauces include but not limit to Dips, Marinades, Yogurts, Guacamole, Salsas, Cheese, Hummus, Sauces, Cream Cheese, Butter, Mustard, Ketchup, Mayonnaise, Apple Sauce, Peanut Butter, etc.

801—exemplifies normal mayonnaise condiments represent in the table as My. 802—exemplifies Enzyme Fortified Mayonnaise (EFF My).

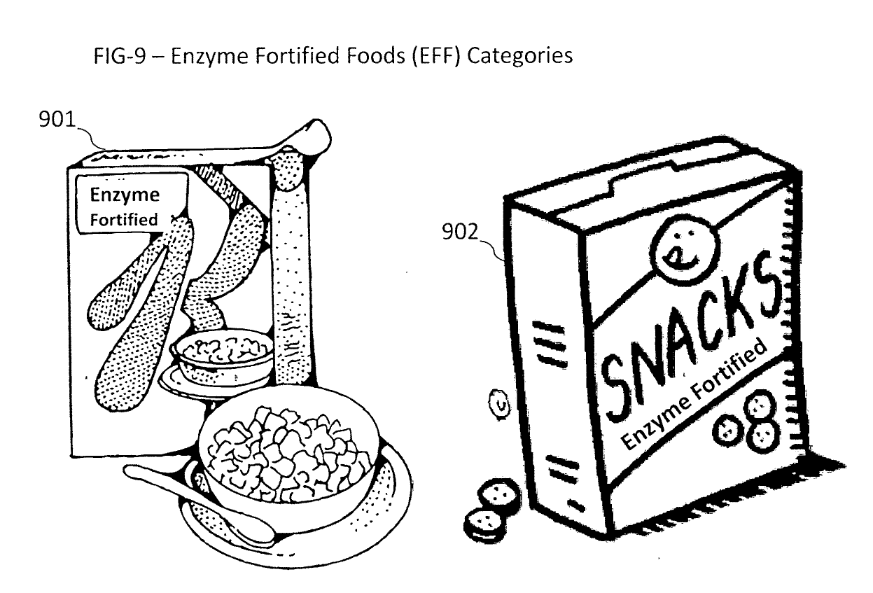

FIG. 9 shows an exemplary of Enzyme enhanced product categories for cereals and snacks, such as 901—EFF cereals, and 902—EFF snacks.

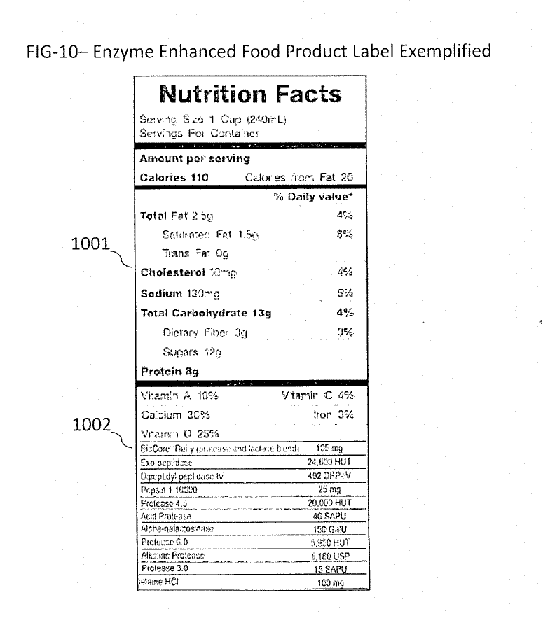

FIG. 10 shows an exemplary of Enzyme enhanced food product label. 1001 shows nutrition facts. EFF could include multi-enzymes, so you'll want to see a number of enzymes listed, 1002—includes proteases 4.5—20,000 HUT, proteases 6.0—5,900 HUT, protease 3.0—15 SAPU (which break down proteins). Look at the labels of the products for specifics—there are a ton of enzymes, but your product should include at least some from these labels.

It should be understood that the steps of the exemplary methods set forth herein are not necessarily required to be performed in the order described, and the order of the steps of such methods should be understood to be merely exemplary. Likewise, additional steps may be included in such methods, and certain steps may be omitted or combined, in methods consistent with various embodiments of the present invention.

As used herein in reference to an element and a standard, the term “compatible” means that the element communicates with other elements in a manner wholly or partially specified by the standard, and would be recognized by other elements as sufficiently capable of communicating with the other elements in the manner specified by the standard. The compatible element does not need to operate internally in a manner specified by the standard.

Also for purposes of this description, the terms “couple,” “coupling,” “coupled,” “connect,” “connecting,” or “connected” refer to any manner known in the art or later developed in which energy is allowed to be transferred between two or more elements, and the interposition of one or more additional elements is contemplated, although not required. Conversely, the terms “directly coupled,” “directly connected,” etc., imply the absence of such additional elements. Signals and corresponding nodes or ports may be referred to by the same name and are interchangeable for purposes here.

It will be further understood that various changes in the details, materials, and arrangements of the parts which have been described and illustrated in order to explain the nature of this invention may be made by those skilled in the art without departing from the scope of the invention as expressed in the following claims.

The invention claimed is:

1. A method of Enzyme Fortified Foods (EFF), the method comprising add-on method: enhance enzyme with food through add-into spices and herbs.

2. The invention of claim 1, wherein EFFs are linked to a person's food intake as a whole and union of various enzymes based on the food itself.

3. The invention of claim 1, wherein EFFs could be customized to any diet, to any food classification, cuisine, combination of intake, or method of fighting any digestive or other disease.

4. A method of Enzyme Fortified Foods (EFF), the method comprising add-on method: enhance enzyme with food mixed with other EFFs when added to vegetables, snacks, pizza, cooked meats, fish, poultry, fruits, etc.

5. The invention of claim 4, wherein EFFs are linked to a person's food intake as a whole and union of various enzymes based on the food itself.

6. The invention of claim 4, wherein EFFs could be customized to any diet, to any food classification, cuisine, combination of intake, or method of fighting any digestive or other disease.

7. A method of Enzyme Fortified Foods (EFF), the method comprising suspended method: enhance enzyme in a suspended substance for frozen goods, beverages, dairy, dehydrated foods, condiments and sauces, (oil, fat, salt sugar), herbs and spices, etc.

8. The invention of claim 7, wherein EFFs are linked to a person's food intake as a whole and union of various enzymes based on the food itself.

9. The invention of claim 7, wherein EFFs could be customized to any diet, to any food classification, cuisine, combination of intake, or method of fighting any digestive or other disease.

10. A method of Enzyme Fortified Foods (EFF), the method comprising coated method: EFF formulated to coat various enzymes through the food manufacturing process without destroying the enzymes.

11. The invention of claim 10, wherein EFFs are linked to a person's food intake as a whole and union of various enzymes based on the food itself.

12. The invention of claim 10, wherein EFFs could be customized to any diet, to any food classification, cuisine, combination of intake, or method of fighting any digestive or other disease.

13. A system for EFF (EnZymed Fortified Foods), the system comprising: Coating Enzyme or Enzyme mixture to food item.

14. A system for EFF (EnZymed Fortified Foods), the system comprising: Mixture to food item.

15. A system for EFF (EnZymed Fortified Foods), the system comprising: additive in addition to food item.

16. A system for EFF (EnZymed Fortified Foods), the system comprising: Embedded to food item.

17. A system for EFF (EnZymed Fortified Foods), the system comprising: Enhancement to food item.

18. A system for EFF (EnZymed Fortified Foods), the system comprising: Seasoning to food item.

19. A system for EFF (EnZymed Fortified Foods), the system comprising: Sprinkling to food item.

20. A system for EFF (EnZymed Fortified Foods), the system comprising: Flavoring to food item.
